(19) United States
(12) Patent Application Publication
(10) Pub. No.: US 2016/0066750 A1
(43) Pub. Date: Mar. 10, 2016

(54) Toothbrush Storage and Sanitizer System

(71) Applicant: Travis Jon Clayton, Auckland (NZ)

(72) Inventor: Travis Jon Clayton, Auckland (NZ)

(21) Appl. No.: 14/812,995

(22) Filed: Jul. 29, 2015

(30) Foreign Application Priority Data
Sep. 9, 2014 (NZ) 700413

(51) Int. Cl. A47K 1/09; B65D 81/22; A61L 2/18; A45D 44/18; A46B 17/06

(52) U.S. Cl. CPC A47K 1/09; A45D 44/18; A46B 17/065; A61L 2/18; B65D 81/22

(57) ABSTRACT

The principal object of this invention is a device to both hold and sanitize toothbrushes when not in use. Keeping toothbrushes both disinfected and pliable will help to preserve oral hygiene and avoid disease. When the device is opened, sanitizer liquid will gravity feed from a container into a connected bath that contains the toothbrush. Because the container is sealed, a vacuum will form, arresting sanitizer flow and resulting in automatic adjustment of the sanitizer level in the bath. Over time the immersed toothbrush will be treated due to contact with the sanitizer. When the device is closed, sanitizer cannot flow out of the container, which allows the device to be easily moved and cleaned. Any number of such devices can be neatly arranged in order to present and sanitize many toothbrushes. The devices will be low cost, potentially disposable, and function without the need for manual refilling or electricity.

### A. BACKGROUND OF THE INVENTION

The principal object of this invention is to provide a low cost, cleanable and effective method of both holding, treating and sanitizing toothbrushes while they are not in use. Keeping toothbrush bristles disinfected will assist to insure proper oral hygiene and avoid disease and infection. Keeping the bristles wet and pliable will reduce the potential for damage to gums from hard bristles. Toothbrush bristles may deliver enhanced performance after immersion in a suitably formulated liquid.

U.S. Pat. No. 1,743,646 has multiple brushes in the same liquid which may introduce cross contamination and furthermore there is no way to stop sanitizer flow or clean the receptacle.

U.S. Pat. No. 6,119,854 has only a small sanitizer pool that has the potential to become contaminated which would require it to be fully discarded and then refilled.

U.S. Pat. No. 3,904,362 has a large sanitizer volume but provides an inconsistent sanitizer liquid level and potential for sanitizer to unnecessarily cover the users handle.

U.S. Pat. No. 4,214,657 makes it difficult to access toothbrushes as the handle is hidden under a removable cap with the handle also in close proximity to other brushes.

U.S. Pat. No. 5,882,613 uses pre-packaged bags of sterilizing solution which would require manual refilling.

U.S. Pat. No. 6,601,699 is more complex and uses air lines and valves and yet still requires manual squeezing to refill the brush reservoir. Furthermore at times this system will deliver excess solution into the toothbrush holder which requires that the system must be tipped over in order to pour out and therefore waste sanitizer solution. The brush holder allows for many orientations of the brush handle which means that the handle alignments are not consistent and neat.

There are many other methods that can be used to sanitize a toothbrush, which do not require a liquid sanitizing solution. These techniques include autoclaving, UV light and exposure to sterilizing gas. The use of such techniques is considered to be undesirable in the usual domestic residential bathroom for a variety of reasons including effectiveness, cost, complexity, hazards and the requirement for electrical power.

While the prior art devices may claim to be suitable for the particular purpose to which they address, they are not in common use due to their shortcomings of function and design. None of the prior art discloses an invention which can adequately sanitize the toothbrush and show all of the combined characteristics of this invention.

### B. SUMMARY OF THE INVENTION

1. A primary object of the present invention is to provide a toothbrush sanitizing and treatment device that will overcome the shortcomings of the prior art devices.

2. A further object of this invention is to a provide a toothbrush sanitizing holder which is both simple and easy in its method of operation so as to increase the usage of toothbrush sanitizing holders among toothbrush users.

3. Another object is to provide a method to keeping the brush bristles wet and pliable.

4. Another object is to make the invention with a small number of parts possible, which will make the invention both low cost, easy to assemble and potentially disposable.

5. A further object is to reduce any mess or spillage of sanitizer through poor bath design or the act of refilling the device.

6. A further object of this invention is to show an automatic refill operation for the brush bath so as to provide a consistent sanitizer liquid level on the toothbrush head.

7. A brush holder that allows the brush handles to be aligned in consistent direction for neatness and ease of access.

8. A further object of this invention is to provide a toothbrush sanitizing holder which prevents cross-contamination by direct contact between the individual toothbrushes.

9. A further object of the invention is to provide a toothbrush sanitizing holder having a sealed reservoir of sterilizing solution of a known formulation and strength.

10. A further object of this invention is for a design of the bath system which can be easily cleaned by closing a valve, in order to purge fouled sanitizer, allow debris in the vessel to be removed and for the vessel to then be cleaned.

In these respects, the brush sanitizer according to the present invention substantially departs from the conventional designs of the prior art, and in so doing provides an apparatus that provides a number of superior functions with a minimum of parts and complexity. Further objects of the invention will appear as the description proceeds.

To the accomplishment of the above and related objects, this invention may be embodied in the form illustrated in the accompanying drawings, attention being called to the fact, however, that the drawings are illustrative only, and that changes may be made in the specific construction illustrated.

### C. DESCRIPTION OF DRAWINGS OF INVENTION

The Applicant has attached four separate sheets, with the following figures of the invention at the end of this patent application:

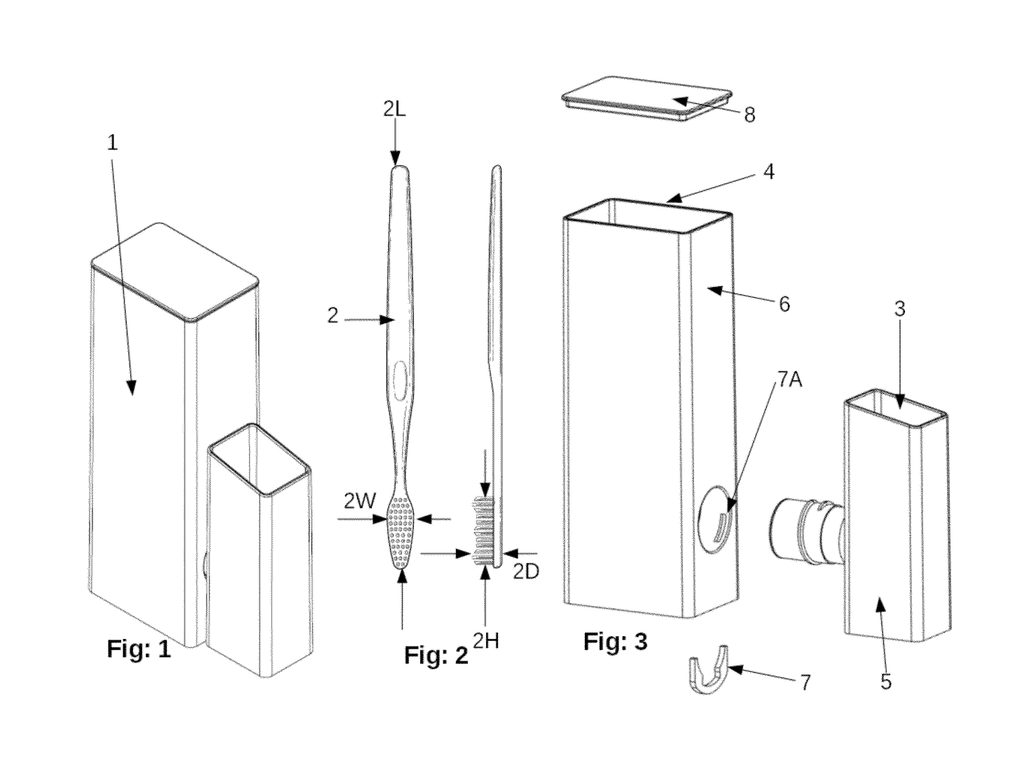

Sheet 1: FIG. 1 is a perspective view of the assembled preferred embodiment.

Sheet 1: FIG. 2 is a side view and plan view of a typical toothbrush showing brush dimensions.

Sheet 1: FIG. 3 is an exploded perspective view of the preferred embodiment showing the method of assembly.

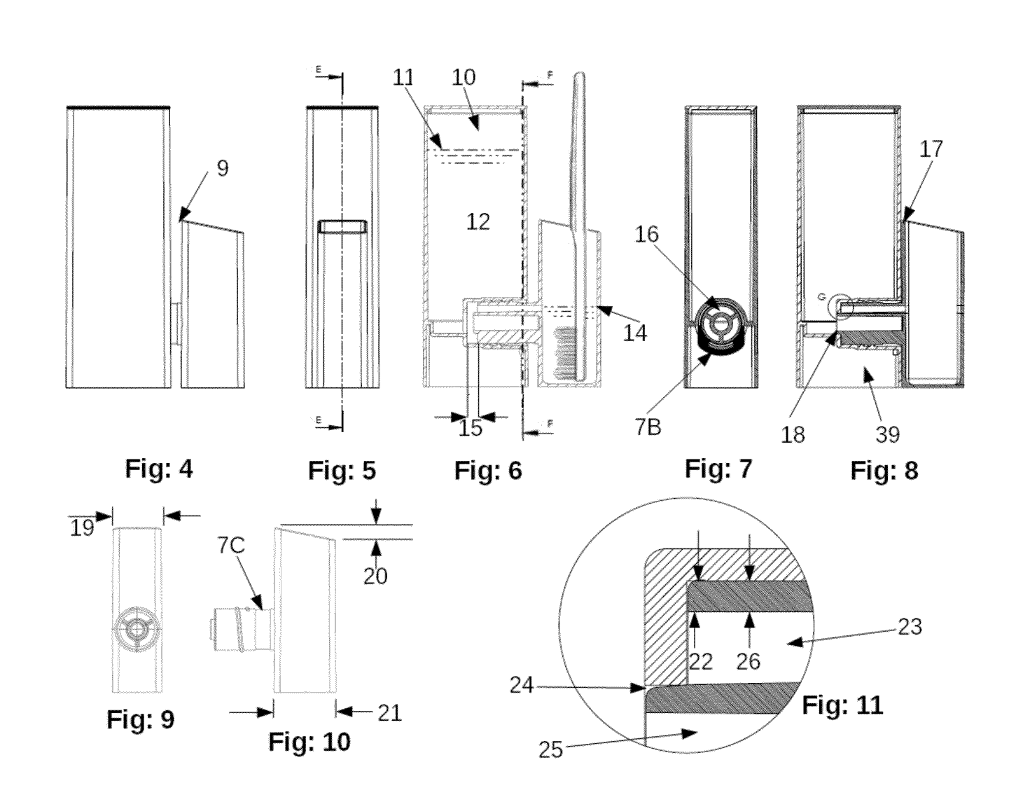

Sheet 2: FIG. 4 is a side view of the preferred embodiment.

Sheet 2: FIG. 5 is a front view of the preferred embodiment.

Sheet 2: FIG. 6 is a sectional view of the preferred embodiment through line EE on FIG. 5.

Sheet 2: FIG. 7 is a sectional view of the preferred embodiment through line FF on FIG. 6.

Sheet 2: FIG. 8 is a sectional view of the preferred embodiment through line EE on FIG. 5.

Sheet 2: FIG. 9 is a rear view of the Bath component used in the preferred embodiment, Part 5 from FIG. 3.

Sheet 2: FIG. 10 is a side view of the Bath component used in the preferred embodiment, Part 5 from FIG. 3.

Sheet 2: FIG. 11 is a detailed side view of the valve area G shown in FIG. 8.

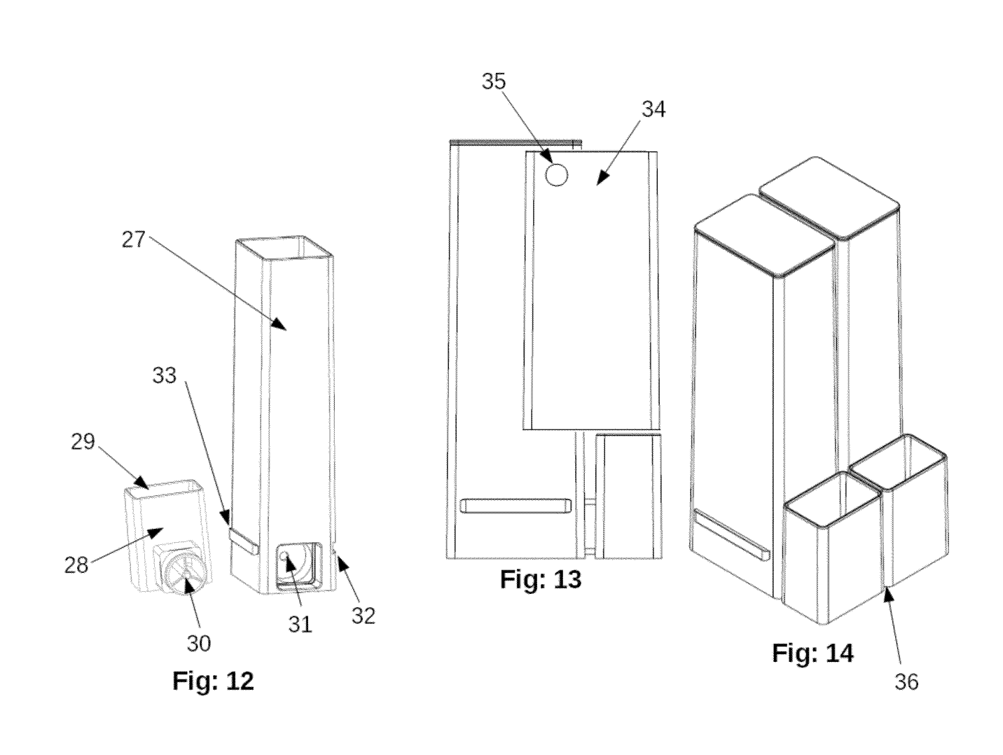

Sheet 3: FIG. 12 is a perspective view of an alternative embodiment.

Sheet 3: FIG. 13 is a side view of an alternative embodiment with a cover.

Sheet 3: FIG. 14 is a perspective view of an alternative embodiment showing two devices attached together.

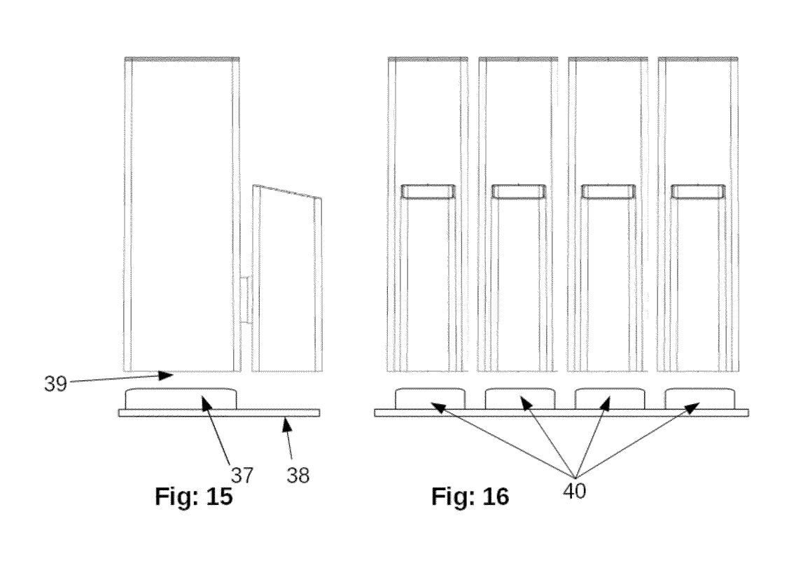

Sheet 4: FIG. 15 is a side view of the preferred embodiment, as well as the side of a plate stand with protrusions.

Sheet 4: FIG. 16 is a front view of four preferred embodiments, as well as a plate stand with protrusions.

### D. DETAILED DESCRIPTION OF INVENTION

This invention, a sanitizing tooth brush holder, is best made from injection moulded plastic parts. Those skilled in the art could manufacture this invention using other materials and processes therefore it is not desired to limit the invention to this exact construction method and material.

FIG. 1: 1 shows the assembled preferred embodiment. FIG. 2 shows a typical Toothbrush 2 and its dimensions. FIG. 3 shows how the component parts of the preferred embodiment are arranged, and then assembled. The Bath 5 would firstly be screwed into the Tower 6 locating the Valve Seal 18 shown in FIG. 8.

The details of the seals are shown in detail in FIG. 11. Barrel Seal 22 is a small protrusion of plastic, whereby the wall thickness shown as 26 is less than the thickness shown as 22. The Barrel seal is used to stop sanitizer from seeping out between Tower 6 and Bath 5 when the sanitizer bath is in use.

Valve Seal 24 is a taper fit between Tower 6 to Bath 5. When the Valve Seal 24 is closed as in FIG. 8 no sanitizer can flow out to the Bath 3. Sanitizer will however be present in area 25 which does not connect with the internal Bath area 3. When the Valve Seal 24 is opened but turning the Bath 360 degrees relative to the Tower, as in FIG. 6 a Gap 15 will be created and allow Sanitizer flow through channel 23 to the internal Bath area 3.

An object of this invention is the unique barrel and valve seal design. Those skilled in the art could manufacture these seals using other methods including “◯” ring or gasket seals or other materials therefore it is not desired to limit the invention to this exact construction method and material.

An alternate embodiment shown in FIG. 12 shows a similar Bath. In this embodiment part 28 would be pressed into part 27 in order to form a Valve seal 18 or 24 by connecting the areas 31 and 30. Therefore the unique internal valve design can be used in either the push fit and screw fit embodiments.

FIG. 3 shows how Clip 7 would then be pressed into holes, 7A shown in Tower 6 and over the internal collar FIG. 10 part 7C. Following correct insertion Clip 7 can be seen to be the dark U shape located in FIG. 7, 7B. This Clip is used to locate the Bath 5 so that it can not be readily separated from the Tower 6 in future. Specifically Clip 7 restricts the movement of the Bath 5 relative to the Tower 6 so that it can not be unscrewed or pulled apart. Those skilled in the art could manufacture this invention using other materials such as a grub screw or other clip design therefore it is not desired to limit the invention to this exact construction method and material.

To be useful the invention must firstly be filled with sanitizing fluid in a manufacturing operation. Once the Valve Seal 24 between the Bath 5 and the Tower 6 has been compressed, the device will be closed and ready to be filled without leaking. This is shown in FIG. 8, whereby Gaps 17 and 18 are reduced.

The Sanitizing liquid 12 is used to the treat toothbrush. The exact determination of such a Sanitizing liquid is not limited in the present embodiment, and may include chemicals that are found to be effective in preventing microbial contamination, such as alcohol or chlorhexidine gluconate. Furthermore the Sanitizing liquid may be formulated to deliver other oral care benefits including but not limited to anti-cavity protection, reduction of dental hypersensitivity, enhanced bristle performance, delivery of materials onto the tooth or gum surface, reduction of plaque or gingivitis or the enhancement of flavour, lubricity or feel.

The filling of Tower 6 would most easily be done through Hole 4 using standard bottling equipment. The sanitizing liquid 12 would be filled into Tower 5 to a Full level 11.

Finally Plug 8 will be used to seal the top of the Tower 6 and contain the fluid for distribution. In order to ensure this invention functions properly, the seal between Plug 8 and Tower 6 will need to be airtight. In the present embodiment the two parts are 8 and 6 are press fitted together with a clip seal around Plug 8 and at point 4 on the Tower 6. Following being clipped together the parts will be ultrasonically welded together, thus it will be difficult to refill Tower 6 or to remove Plug 8 from the top due to the melted plastic. Those skilled in the art could manufacture this seal using other methods and processes therefore it is not desired to limit the invention to this exact construction method. The seal could also be achieved by using other standard methods including threaded seal, push fit, glue or gaskets. If a threaded design were used to seal between part 8 and part 6 the invention could become reusable as the Tower 6 could be separated from Plug 8 and then refilled.

Once the tower is filled it can be distributed to the consumer. In the present embodiment the consumer can not refill the container easily because Clip 7 is located around 7c and through Tower 6 at the two Holes 7A. This will make it difficult to separate bath 5 from Tower 6.

To use the invention the sanitizer system needs to be opened by the consumer. To achieve this Bath 5 needs to be moved to create a gap FIG. 6: 15. The Gap FIG. 4: 9 between Bath 5 and Tower 6 is also increased. In the preferred embodiment the consumer will simply rotate the Bath 5, 360 degrees relative to Tower 6 in order to open the Valve Seal 24. In the alternate embodiment FIG. 12, Bath 28 could be simply pulled in order to separate it from Tower 27 and open the Valve Seal 24. These Valve seal connections are also shown as 30 and 31.

Performing either of these actions will create the Gap FIG. 6: 15. This gap will allow air from outside the container to enter into Tower 6 through Entry 3 and Barrel FIG. 7: 16. In detail FIG. 11 the path of flow of air will be through Channel 23 past Valve Seal 24 and into Tower 6. The sanitizing liquid will then flow out through the same path, creating a Vacuum 10 in Tower 6. The sanitizer will continue to flow out until it reaches Level 14.

If Plug 8 was not fitted with an airtight seal at the top of the Tower, point 4, there would be no vacuum and the sanitizer would fully empty from the Tower 6. Because the Tower 6 is not vented, a vacuum is created inside the tower so the sanitizer stays in the Tower until the level in the bath goes down far enough to uncover the internal Aperture FIG. 12: 31 or FIG. 11: 24. Once this aperture is vented the level in the bath fills up again to cover the Aperture. Thus a consistent liquid Level 14 is maintained within the brush bath. This system will continue to operate until the Tower liquid Level 11 is equal to the Bath liquid Level 14.

Typically other inventions are variations of simple cup like containers or inverted bottle dispensers. This invention is different from other inventions in that the liquid is introduced into the chamber through one aperture and out to sanitize brushes through a different aperture in the package. Those skilled in the art may fix Tower 6 to Plug 8 firstly and then attempt to fill Tower 6 using Aperture 31, therefore it is not desired to limit the invention to which aperture the tower is filled through. This invention is unique in that the multiple aperture system has advantages for both manufacturing and operation that should now be apparent.

The following mathematical relationships are used. In FIG. 2 the Width of the head of a toothbrush is 2W, the Height of the toothbrush bristles is 2H, the Depth of the bristles is 2D and the length of the entire brush is 2L. Once opened the user can insert the Toothbrush Head with dimensions FIG. 2: 2W, 2H, 2D into the Opening 3 in Bath 5. The head of the brush will sit below the liquid Level 14. After after a suitable time the toothbrush will be sanitized.

Generally the Depth of toothbrush bristles 2D is greater than the Width of the toothbrush 2W. Because of this a component of this invention is to use a Bath that has dimensions such that the internal Bath width FIG. 9 19 is less than brush head Bristle depth 2D. This design ensures that the brush will typically fall forwards.

In the preferred embodiment the height of Tower 6 is less than the Length of the toothbrush 2L so that the toothbrush handle extends beyond the tower and is therefore easily grabbed by the user.

An object of this invention is to provide an organised sanitizer whereby the toothbrushes are neatly arranged. An important relationship relating to this is the difference in height between the front of Bath 5 and the rear Distance 20 in FIG. 10. The Difference 20 in height ensures that the brush will typically fall forwards.

A benefit of these many design considerations is that the tooth brush handle can be easily grabbed by the consumer without having the brush handles cross each other or assume variable orientations. This occurs because of the relationships explained above ensure that a toothbrush can only be located either facing forward or backwards relative to the bath, and the handle will lean forward so can be easily grabbed. The alternate embodiment Bath FIG. 12: 28 does not have all of these features. As a result a brush located into this bath can be rotated and thus the handle is not neatly aligned so it can fall in any direction forwards, backwards or sideways.

Also of significance in the preferred embodiment is that the Bath length FIG. 10: 21 is greater than the brush head Bristle height FIG. 2: 2D. This ensures that the brush will fit into the bath.

The sanitizer depth 14 is greater the Brush head height FIG. 2: 2H. In this way all of the brush bristles will be covered in sanitizer.

Over time the sanitizer volume in the Bath 5 will be reduced. The sanitizer reduces when a consumer uses the brush and removes drops of sanitizer on the bristles. Further sanitizer will be lost due to evaporation. Automatically the sanitizer level in Bath 5 will equilibrate to Level 11 due to the balance between the Vacuum 10, the height of the Sanitizer 12 in the tower and atmospheric pressure.

After some time in operation the internal area 3 of Bath 5 may become soiled with debris including food scraps or other contaminants. In this case the Bath 5 can be cleaned by lifting the entire device, and in the case of the preferred embodiment rotate Bath 5 in order to close Valve Seal 18. By doing this the sanitizer in Bath 5 can be tipped or drained with a small amount of treatment liquid released in order to purge Bath 5. The bath can then be rinsed under a tap and the internal area brushed or similar in order to clean it. Following this cleaning procedure the bath can be placed back into service by opening Valve 18 in order to fill the bath to Level 14 again.

An alternative embodiment FIG. 13 shows a cover that can be used to protect the bath Opening 3 and toothbrush from airborne particulates or contaminants. This embodiment has been shown with Cover 34. An example Hinge 35 can be used to attach the cover and allow it to swing open to allow brush access.

Another embodiment is shown in FIG. 15. A Plate Stand 38 with multiple raised Protrusions 37 and 40 which can be used to locate multiple devices together in a neat fashion. The Protrusions 37 fit under and into the tower Base FIG. 8: 39 in order to lock them together and limit movement. This arrangement also allows any device to be easily lifted and removed for individual cleaning or replacement.

A further alternative embodiment FIG. 14 shows how multiple baths can be joined together in order to form a neat holder for multiple brushes. FIG. 12 shows a Key 33 on the side of Tower 27. This key locates into a matching Keyhole 32 on the other side of the tower. This key arrangement can be aligned horizontally or vertically in order to fix and locate the devices together, with little gap FIG. 14: 36.

Alternative embodiments may use the core design principles of this design for the treatment of other implements or devices other than a toothbrush. Specifically the invention described can be used with minimal modifications to treat different tools, razors, implements or brushes with differing formulations of sanitizer liquid.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents maybe resorted to, falling within the scope of the invention.

1. A device suitable for the treatment of toothbrushes as shown and described comprising;
a) a tower composed of rigid material to hold a volume of sanitizer liquid,
b) at least one aperture in the said tower which allows the said tower to be filled with liquid,
c) at least one aperture in the said tower which allow liquid to flow out,
d) a bath container composed of a rigid material such that it can hold a volume of sanitizer liquid and a toothbrush head,
e) an aperture in the said bath of a sufficient size to accept a toothbrush,
f) at least one aperture in the said bath that allows liquid to enter into the said bath,
g) a valve seal located between the bath and the tower, which when opened allows liquid to flow between the tower and the bath, and when it is closed will prevent liquid flow,
h) a barrel seal located between the bath and the tower, which prevents liquid from escaping, or air from entering, the device,
i) a plug composed of a rigid material of sufficient size attached to the said tower with an air tight seal.

2. The device of claim 1, further comprising a clip composed of rigid material attached to the said tower and the said bath, thus stopping the two from being separated.

3. The device of claim 1, further comprising that the said bath has a width less than the depth of a toothbrush, so that a toothbrush cannot be freely rotated when it is in the bath.

4. The device of claim 1, further comprising that the said bath has dimensions such that the front of the bath is lower than the rear of the bath.

5. The device of claim 1, further comprising a plate stand composed of rigid material having a plurality of protrusions, such that a plurality of the said devices can be located onto the said protrusions on the said base thus restricting the plurality of devices from free movement.

6. The device of claim 1, further comprising a plurality of keys and a plurality of matching keyholes such that a plurality of the devices can be attached together using the said keys and the said keyholes.

7. The device of claim 1, further comprising at least one cover which acts to prevent airborne material from entering the said bath.
